(19) United States
(12) Patent Application Publication
(10) Pub. No.: US 2025/0213759 A1
(43) Pub. Date: Jul. 3, 2025

(54) LEAKAGE-PROOF WEARABLE BREAST PUMP

(71) Applicant: Dongguan Rongfeng Medical Co., Ltd., Dongguan (CN)

(72) Inventor: Yongqiang ZHANG, Fuyang (CN)

(21) Appl. No.: 18/424,377

(22) Filed: Jan. 26, 2024

(30) Foreign Application Priority Data
Dec. 30, 2023 (CN) 202323670933.5

(51) Int. Cl. A61M 1/06; A61M 1/00

(52) U.S. Cl. CPC A61M 1/062; A61M 1/80; A61M 2205/02; A61M 2205/07; A61M 2210/1007

(57) ABSTRACT

A leakage-proof wearable breast pump includes a milk bottle, a front cover, and a negative pressure chamber, the front cover and the negative pressure chamber are both arranged above the milk bottle and a connecting chamber is provided therebetween; the connecting chamber is provided above the bottle mouth of the milk bottle, a buffer chamber is provided between the milk bottle and the connecting chamber, and the buffer chamber is communicated with the milk bottle and the connecting chamber respectively. The user wears the breast pump to move, the liquid surface in the milk bottle will shake, thereby overflowing the bottle mouth or the vent hole. The buffer chamber can collect the milk overflowing and reflow it, thereby preventing leakage.

### CROSS-REFERENCE TO RELATED APPLICATION

This application claims priority from the Chinese patent application 202323670933.5 filed Dec. 30, 2023, the content of which is incorporated herein in the entirety by reference.

### TECHNICAL FIELD

The present disclosure relates to the technical field of breast pumps, more particularly, a leakage-proof wearable breast pump.

### BACKGROUND

Breast bump generally includes negative a pressure pump, a negative pressure chamber, a flange, and a milk bottle. The negative pressure pump is generally connected with the negative pressure chamber through a three-way valve. The negative pressure pump repeatedly “inhales” and “exhales” to simulate the action of a baby sucking breast, to achieve the purpose of breast milk secretion. The wearable breast pump can satisfy the object of the user to secret, collect, and store the milk in the bottle when the user moves or in public, which can satisfy the demand of the majority of workplace mothers.

The current breast pump is provided with a one-way valve at the bottle mouth of the milk bottle to drain the milk into the milk bottle. The top of the milk bottle is provided with a vent hole to expel air from the milk bottle, thereby maintaining a balance between the air pressure in the bottle and the external air pressure.

However, in practical use, the milk flowing into the milk bottle often overflows through the vent hole. This is mainly because a user moves when wearing a breast pump, if there is a lot of milk in the milk bottle, the surface of the milk shakes in a wavy shape, and the milk is pushed to overflow from the vent hole when the air in the milk bottle is expelled normally. Alternatively, the user tilts her upper body or bends down when wearing a breast pump, causing the vent hole originally facing upward to tilt relative to the horizontal plane, thereby the milk in the milk bottle leaking out from the vent hole. Alternatively, when the milk enters a milk bottle, it impacts the bottle wall or the milk in the bottle, splashes up some droplets, and falls into the vent hole and overflows.

### SUMMARY

The present disclosure provides a leakage-proof wearable breast pump to solve the problem that a wearable breast pump in the prior art easily leaks milk from a vent hole.

The technical schemes of the present disclosure are as follows: a leakage-proof wearable breast pump includes a milk bottle, a front cover, and a negative pressure chamber, the front cover and the negative pressure chamber are both arranged above the milk bottle and a connecting chamber is provided therebetween; the connecting chamber is provided above the bottle mouth of the milk bottle, a buffer chamber is provided between the milk bottle and the connecting chamber, and the buffer chamber is communicated with the milk bottle and the connecting chamber respectively.

Preferably, the top surface of the milk bottle extends upwards around the bottle mouth to form the buffer chamber; the bottom of the connecting chamber is provided with a cover plate to cover the buffer chamber; the cover plate is provided with a one-way valve, directly facing the bottle mouth.

Or, the bottom of the connecting chamber is provided with the cover plate, the cover plate extends downward to form the buffer chamber, the buffer chamber surrounds the bottle mouth; the cover plate is provided with the one-way valve directly facing the mouth of the bottle mouth.

Or, the bottom of the buffer chamber is connected with the top of the milk bottle and surrounds the bottle mouth, and the top of the buffer chamber is connected to the cover plate disposed on the bottom of the connecting chamber, and the cover plate is provided with the one-way valve, facing the bottle mouth.

The plate cover is provided with a vent hole in communication with the buffer chamber and the outside.

Preferably, the end of the one-way valve is close to or extends into the bottle mouth, and a gap is provided between the end of the one-way valve and an inner wall of the bottle mouth.

Preferably, the leakage-proof wearable breast pump further includes a mounting base with a bottom plate, the bottom plate is provided on the top of the milk bottle, the bottom plate is provided with an arch portion, and the connecting chamber and the buffer chamber are installed up and down in the arch portion.

Preferably, the connecting chamber is provided with a guiding cylinder attached to the arch portion, and the bottom of the arch portion is provided with an arc-shaped part, attached to a side wall of the buffer chamber so as to protect the buffer chamber.

Preferably, the front cover is mounted on the mounting base, with a shape to cooperate with the front side surface of the milk bottle to form a horn chamber; a small-diameter end of the horn chamber is formed on the front cover and inserted into the arch portion, close to or tightly adhered to one end of the guiding cylinder.

Preferably, the negative pressure chamber includes a corrugated piece, a rear baffle, and a front baffle disposed at the other end of the guiding cylinder; the corrugated piece is made of a flexible silica gel material and is arranged in a groove shape, comprising a corrugated portion forming the bottom of the groove and the side portion forming the side wall of the groove; a joint of said rear baffle and said front baffle clamps said side portion of said corrugated piece, thereby sealing a cavity between the rear baffle and the front baffle, and installing the corrugated portion in the negative pressure chamber.

Preferably, the front baffle is provided with at least one hole, and the negative pressure chamber is communicated with the connecting chamber and the horn chamber.

Preferably, the breast pump further includes a negative pressure pump, the negative pressure pump includes an air pump, a solenoid valve, and a three-way valve; the three-way valve is disposed on the mounting base, and the three air passages of the three-way valve are respectively in communication with the negative pressure chamber, the air pump and the solenoid valve.

Preferably, the breast pump further includes a rear cover mounted on the mounting base and its shape is matched with the rear side of the milk bottle to form an arc-shaped surface.

The advantages of the technical schemes of the present disclosure are as follows:

In the leakage-proof wearable breast pump, the buffer chamber is disposed between the milk bottle and the connecting chamber. The first function of the buffer chamber is as follows: if the milk in the milk bottle is relatively full, the user wears the breast pump to perform actions such as walking, rotating, etc., the milk in the milk bottle will shake in a wave shape, thereby overflowing the vent hole or the bottle mouth of the milk bottle. The buffer chamber can collect the milk flowing out, thereby preventing leakage. The second function of the buffer chamber is as follows: in normal conditions, the user stands upright, and the vent hole or the bottle mouth of the milk bottle faces upward. When the user inclines her upper body or bends her waist, the milk bottle will incline accordingly, and if there is a lot of milk in the milk bottle at this moment, the milk will flow out of the vent hole or the bottle mouth, and the buffer chamber can collect the flowing-out milk and reflow it into the milk bottle to prevent the milk from overflowing out of the milk bottle. The third function of the buffer chamber is as follows: when the milk flows into the milk bottle, the milk impacts the wall or the liquid surface, splashes up some droplets, partially falls on the top wall of the milk bottle, and flows backward, and partially enters the buffer chamber and flows backward to the milk bottle, thereby preventing the milk from overflowing.

The leakage-proof wearable breast pump disclosed in the present disclosure, the vent hole is disposed at the side wall of the buffer chamber or the cover plate, thereby preventing the milk from overflowing from the vent hole, and keeping the vent hole unobstructed, so that the interior and exterior of the milk bottle are balanced in atmospheric pressure.

### BRIEF DESCRIPTION OF THE DRAWINGS

FIG. 1 is a perspective view of the leakage-proof wearable breast pump according to the present disclosure.

FIG. 2 is an exploded view of the leakage-proof wearable breast pump shown in FIG. 1.

FIG. 3 is a perspective view of the milk bottle, the buffer chamber and the connecting chamber according to the present disclosure.

FIG. 4 is a front view of FIG. 3.

FIG. 5 is an in-sectional view along A-A line in FIG. 3.

FIG. 6 is an exploded view of FIG. 5.

FIG. 7 is an in-sectional view along B-B line in FIG. 3.

FIG. 8 is a perspective view of the leakage-proof wearable breast pump removing the front cover, the rear cover, the top cover, and the battery according to the present disclosure.

FIG. 9 is a perspective view from another angle of FIG. 8.

FIG. 10 is a front view of the leakage-proof wearable breast pump according to the present disclosure.

FIG. 11 is an exploded view along C-C line in FIG. 10.

The reference number: The front cover 10, the rear cover 20, the top cover 30, the display screen 31, the switch button 32, the control button 33, the flange 40, the battery 60, the PCB circuit board 70, the milk bottle 100, the bottle mouth 101, the gap 102, the front side surface 103, the top wall 110, the main body 120, the horn chamber 130, the small-diameter end 131, the sealing ring 140, the negative pressure chamber 200, the corrugated piece 210, the side portion 211, the corrugated portion 212, the rear baffle 220, the front baffle 230, the hole 231, the connecting chamber 300, the cover plate 310, the through hole 301, the side wall 311, the sliding groove 312, the snapping groove 313, the vent hole 314, the one-way valve 320, the guiding cylinder 330, the buffer chamber 400, the boss 410, the mounting base 500, the the bottom plate 510, the arch portion 520, the arc-shaped part 521, the negative pressure pump 600, the air pump 610, the solenoid valve 620, the three-way valve 630, the air passage 631, 632 and 633.

### EMBODIMENTS

To make the objectives, technical solutions, and technical effects of the present disclosure clearer and more comprehensible, the following further describes the present disclosure with reference to embodiments. It should be understood that the embodiments described herein are only used to explain the present disclosure, but not used to limit the present disclosure.

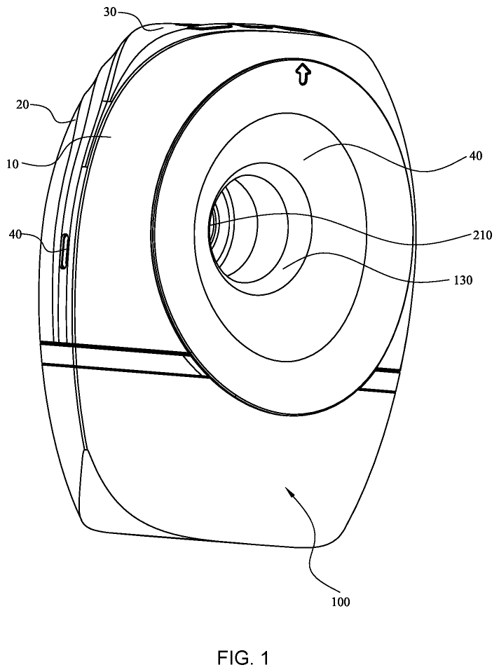

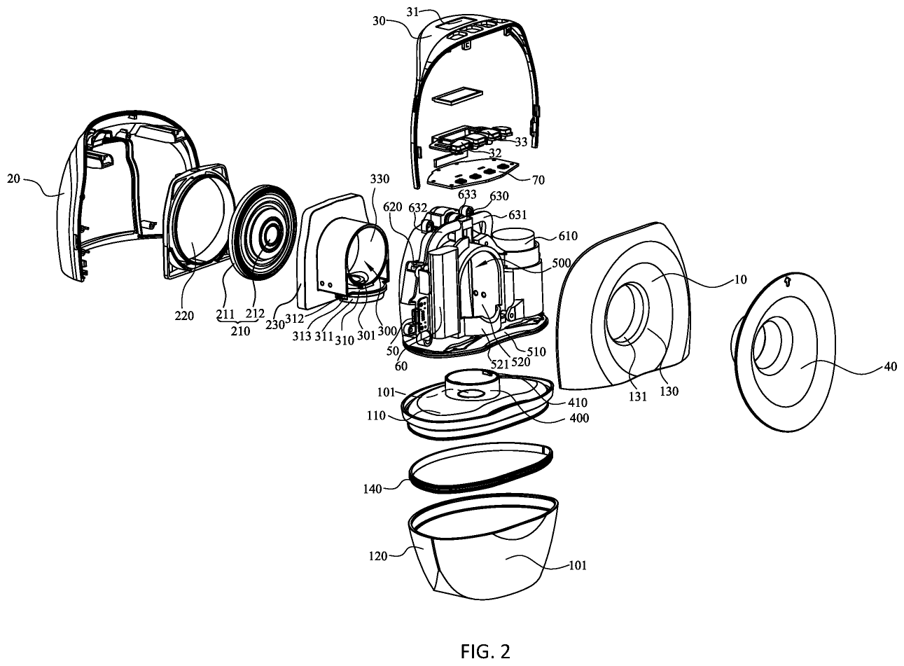

Referring to FIGS. 1-2, a leakage-proof wearable breast pump includes a milk bottle 100, a front cover 10, and a negative pressure chamber 200. The front cover 10 and the negative pressure chamber 200 are both provided above the milk bottle 100 and a connecting chamber 300 is provided therebetween. The connecting chamber 300 is provided above the bottle mouth 101 of the milk bottle 100. A buffer chamber 400 is provided between the milk bottle 100 and the connecting chamber 300, and the buffer chamber 400 is communicated with the milk bottle 100 and the connecting chamber 300 respectively. The milk secreted by a mother flows through the front cover into the connecting chamber 300, then flows through the buffer chamber 400 into the milk bottle 100.

The first function of the buffer chamber 100 is as follows: if the milk in the milk bottle 100 is relatively full, the user wears the breast pump to perform actions such as walking, rotating, etc., the milk in the milk bottle 100 will shake in a wave shape, thereby overflowing the vent hole or the bottle mouth of the milk bottle 101. The buffer chamber 400 can collect the milk flowing out, thereby preventing leakage. The second function of the buffer chamber 400 is as follows: in normal conditions, the user stands upright, and the vent hole or the bottle mouth 101 of the milk bottle 100 faces upward, When the user performs an action such as inclining his or her upper body or bending his or her waist, the milk bottle 100 will incline accordingly, and if there are a lot of milk in the milk bottle 100 at this moment, the milk will flow out of the vent hole or the bottle mouth 101, and the buffer chamber 400 can collect the flowing-out milk and reflow it into the milk bottle 100 to prevent the milk powder from overflowing out of the milk bottle 100. The third function of the buffer chamber 400 is as follows: when the milk flows into the milk bottle 100, the milk impacts the wall or the liquid surface, splashes up some droplets, partially falls on the top wall 110 of the milk bottle 100, and flows backward, and partially enters the buffer chamber 400 and flows backward to the milk bottle 100, thereby preventing the milk from overflowing.

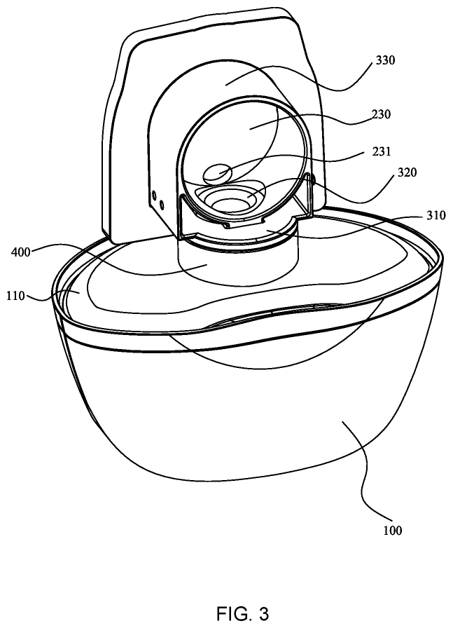

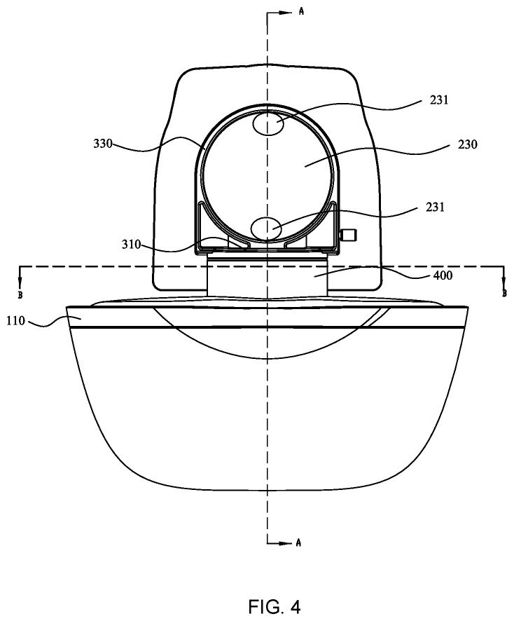

It should be understood that the buffer chamber can be configured in many manners, in an embodiment, referring to FIGS. 3-5, the top surface of the milk bottle extends upwards around the bottle mouth 101 to form the buffer chamber 400. The bottom of the connecting chamber 300 is provided with a cover plate 310 to cover the buffer chamber 400. The cover plate 310 is provided with a one-way valve 320, directly facing the bottle mouth 101. The milk flows into the connecting chamber 300, passes through the one-way valve 320, and the bottle mouth 101, and is collected in the milk bottle 100. In an arrangement manner, the bottom of the buffer chamber 400 is integrally formed with the top of the milk bottle 100, and the top of the buffer chamber 400 is connected to the cover plate 310 a snap-fit manner or in a threaded manner.

Specifically, referring to FIG. 2, the inner wall of the buffer chamber 400 is provided with a boss 410, the cover plate 310 extends downwards to form a side wall 311, an outer wall of the side wall 311 is provided with a sliding groove 312, and an end of the sliding groove 312 is provided with a snapping groove 313. Rotating the buffer chamber 400, the boss 410 is slid into the snapping groove 313 along the sliding groove 312 and clamped therein. Or reversely rotating the buffer chamber 400, the boss 410 is reversely slid along the sliding groove 312 to disengage from the snapping groove 313, thereby disassembling the milk bottle 100 and the cover 310.

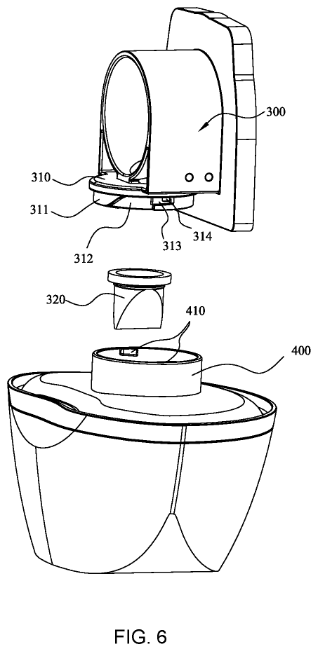

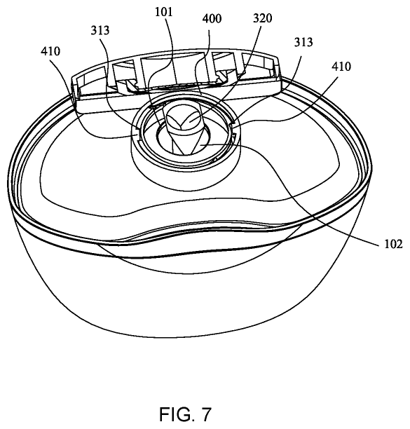

Referring to FIG. 2, FIGS. 6-7, the plate cover 310 is provided with a vent hole 314 in communication with the buffer chamber 400 and the outside, that is in communication with the interior of the milk bottle 100 and the outside, thereby maintaining the air pressure balance inside and outside the milk bottle 100. The vent hole 314 is preferably provided on the side wall 311 of the cover plate 310. Preferably, the vent hole 314 is provided on the bottom surface of the snapping groove 313. When the boss 410 is accommodated in the snapping groove 313, a gap is between the boss 410 and the snapping groove 313 so that air can pass through the vent hole 310 and the gap between the boss 410 and the snapping groove 313 to flow between the buffer 400 and the outside, that is to maintain the communication between the bottle 100 and the outside. In addition, the vent hole 314 is placed in the bottom surface 313 of the snapping groove 313, when the milk flows into the vent hole 314, the boss 410 can reduce the flow rate to a certain extent, and reduce overflow and leakage as far as possible.

In an embodiment, the bottom of the connecting chamber 300 is provided with the cover plate 300, the cover plate extends downward to form the buffer chamber 400, the buffer chamber 400 is around the mouth of the bottle 101. The cover plate 310 is provided with the one-way valve 320 directly facing the mouth 101 of the bottle. In the arrangement manner, the top of the buffer chamber 400 is integrally formed with the cover plate 310, the bottom of the buffer chamber 400 is connected with the bottle in a snap-fit manner or a threaded manner.

In the embodiment, the bottle mouth 101 is preferably an annular structure extending upwards from the top surface of the milk bottle. The buffer chamber 400 and the bottle mouth 101 are also connected in a snap-fit connection or a thread connection as in the described embodiment. The structure of the snap-fit connection and the vent holes are the same as those in the described embodiment, and will not be repeated here. The vent holes are formed in an upper portion of a side wall of the buffer chamber 400.

In another embodiment, the bottom of the buffer chamber 400 is connected with the top of the milk bottle 100 and surrounds the bottle mouth 101. And a top of the buffer chamber 400 is connected to the cover plate 310 disposed on the bottom of the connecting chamber 300. The cover plate 310 is provided with the one-way valve 320, facing the bottle mouth 101. In an arrangement manner, the bottom of the buffer chamber 400 is connected to the milk bottle 100 in a threaded connection or a snap-fit connection manner, and the top of the buffer chamber 400 is connected to the cover plate 300 in a snap-fit connection or a threaded connection manner. The structure of the snap-fit connection and the exhaust holes are the same as those in the described embodiment, and will not be elaborated herein.

Referring to FIG. 7, the end of the one-way valve 320 is close to or extends into the bottle mouth 101, and a gap 102 is provided between the end of the one-way valve 320 and an inner wall of the bottle mouth 101 so that air can be conveniently discharged out of the milk bottle 100.

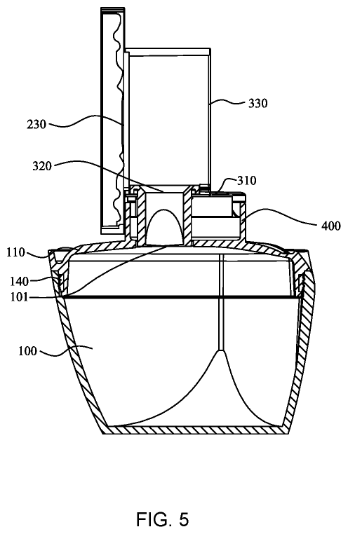

The top 110 of the milk bottle 100 and the main body 120 of the milk bottle 100 are integrally formed, with this arrangement manner, the milk bottle 110 facilitates production and reduces installation steps, thereby saving costs. Referring to FIGS. 2 and 5, the top 110 of the milk bottle 100 and the main body 120 of the milk bottle 100 can be separately provided, with this arrangement manner, the milk bottle 100 is easy to clean. A sealing ring 140 is arranged at the join of the top 110 of the milk bottle 100 and the main body 120 of the milk bottle 100, thereby sealing the milk bottle and preventing leakage.

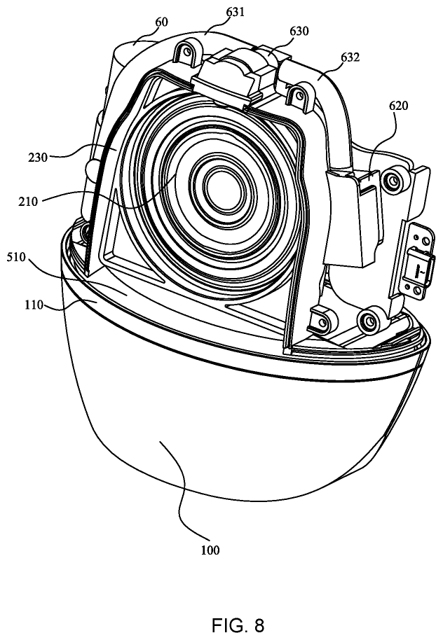

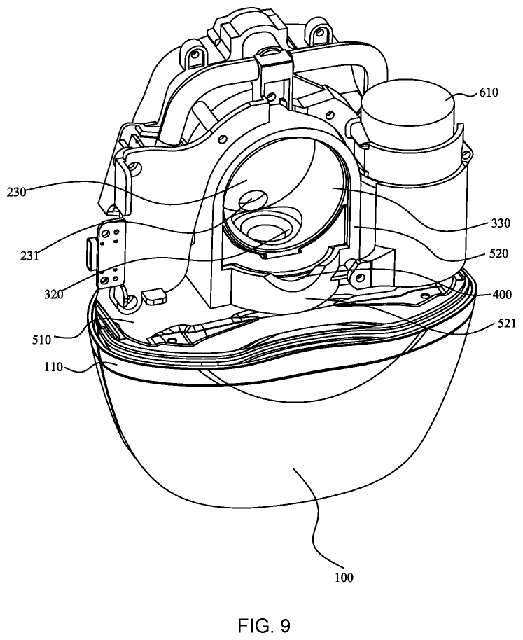

Referring to FIGS. 2, 8, and 9, the top of the milk bottle is provided with a mounting base 500, the mounting base 500 is provided with a bottom plate 510, and the bottom plate 510 is arranged on the top 110 of the milk bottle 100. The bottom plate 510 is provided with an arch portion 520, and the connecting chamber 300 and the buffer chamber 400 are installed up and down in the arch portion 520. Specifically, the connecting chamber 300 is provided with a guiding cylinder 330 and the cover plate 310, and the guiding cylinder 330 is attached to the arch portion 520. The cover plate 310 is disposed below the guiding cylinder 330 and covers the buffer chamber 400. the bottom of the arch portion 520 is provided with an arc-shaped part 521, attached to a side wall of the buffer chamber 400 so as to protect the buffer chamber 400. The connecting chamber 300 is provided with a through hole 301 penetrating through the guiding cylinder 330 and the cover plate 310, and the one-way valve 320 is installed at the through hole 301.

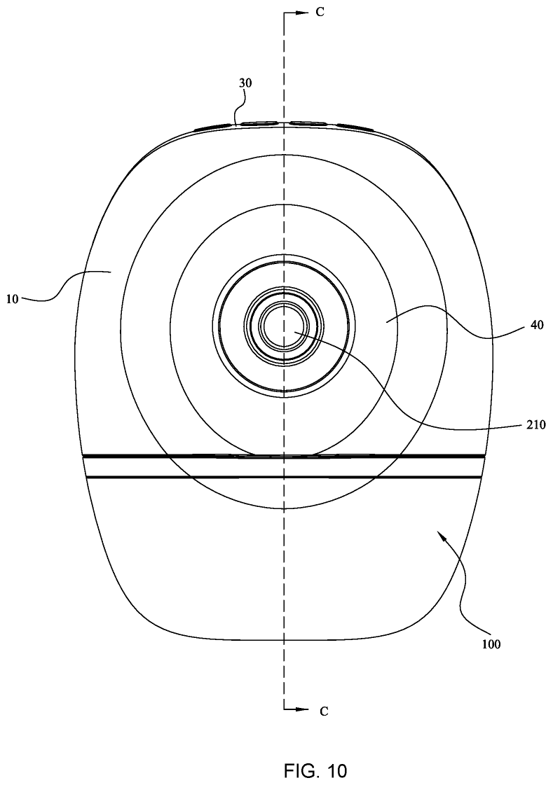

Referring to FIGS. 1, 10, and 11, the front cover 10 is mounted on the mounting base 500, with a shape to cooperate with the front side surface 103 of the milk bottle 100 to form a horn chamber 130, and a flange 40 is attached to the horn chamber 130. The flange 40 is made of a flexible material, such as silica gel, so that the user feels soft and comfortable and the horn chamber 130 can be sealed when the flange 40 fits the breast. A small-diameter end 131 of the horn chamber 130 is formed on the front cover 10 and inserted into the arch portion 520, close to or tightly adhered to one end of the guiding cylinder 330. The secreted milk flows through the horn chamber 130 formed in the front cover 10, and into the guiding cylinder 330, the one-way valve 320, the buffer chamber 400, and the milk bottle 100 in sequence.

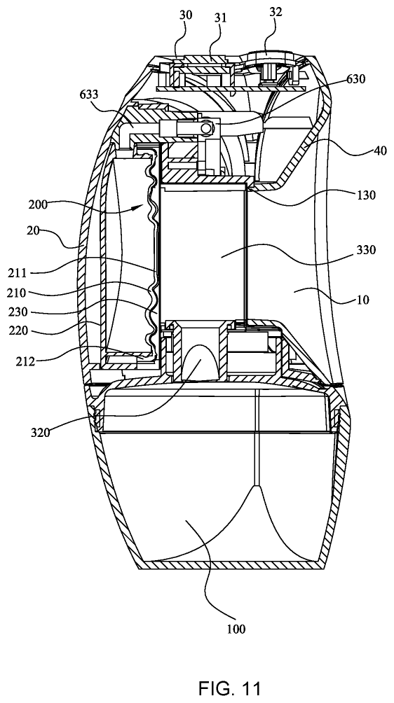

Referring to FIGS. 2 and 11, the negative pressure chamber 200 includes a corrugated piece 210, a rear baffle 220, and a front baffle 230 disposed at the other end of the guiding cylinder 330. The corrugated piece 210 is made of a flexible silica gel material and is arranged in a groove shape, comprising a corrugated portion 212 forming the bottom of the groove and a side portion 211 forming the side wall of the groove. The rear baffle 220 and the front baffle 230 are connected, and a joint between the rear baffle 220 and the front baffle 230 clamps the side portion of the corrugated piece 210, thereby sealing a cavity between the rear baffle 220 and the front baffle 230, and installing the corrugated portion 212 in the negative pressure chamber 200.

Referring to FIGS. 3, 4, and 9, the front baffle 230 is provided with at least one hole 231, namely the negative pressure chamber 200 is communicated with the connecting chamber 300 and the horn chamber 130 formed in the front cover 10 through the hole 231.

Referring to FIGS. 2, 8, and 9, the breast pump further includes a negative pressure pump 600, which is communicated with the negative pressure chamber 200, by means of repeatedly “inhaling” and “exhaling” to the negative pressure chamber 200 to simulate the action of a baby sucking breast, to achieve the purpose of breast milk secretion. Specifically, when the breast pump is used, the flange 40 tightly fits the mother's breast to seal the horn chamber 130 formed by the front cover 10, and the one-way valve is closed, then the negative pressure chamber 200, the horn chamber 130 and the connecting chamber 300 jointly form a sealed space. The negative pressure pump 600 inhales from the negative pressure chamber 200, the air in the horn chamber 130 and the connecting chamber 300 flows through the hole 231 into the negative pressure chamber 200 and is inhaled by the negative pressure pump 600, thereby forming a negative pressure in the sealed space. The negative pressure pump 600 exhales to the negative pressure chamber 200, then air enters the negative pressure chamber 200 and flows through the hole 231 into the connecting chamber 300 and the horn chamber 130, thereby forming a positive pressure in the sealed space.

When the negative pressure pump 600 inhales or exhales, the corrugated piece 210 elastically expands, and the rear baffle 220 and the front baffle 230 have the effect of preventing the corrugated piece 210 from expanding excessively.

The negative pressure pump 600 includes an air pump 610, a solenoid valve 620, and a three-way valve 630. The three-way valve 630 is disposed on the mounting base 500, and the three air passages 631, 632, and 633 of the three-way valve 630 are respectively in communication with the negative pressure chamber 200, the air pump 610 and the solenoid valve 620. As shown in FIG. 2, the air pump 610 and the solenoid valve 620 are separately mounted on the mounting base 500 and located at two sides of the arch portion 520.

Referring to FIGS. 1-2, the breast pump also includes a rear cover 20 mounted on the mounting base 500 and its shape is matched with the rear side of the milk bottle 100 to form an arc-shaped surface, thereby making the breast pump have a beautiful shape.

The breast pump further includes a top cover 30, arranged between the top of the rear cover 20 and that of the front cover 10, the top cover 30 is provided with a display screen 31, a switch button 32, and a control button 33.

The breast pump also includes a PCB circuit board 70 and a battery 60. The PCB circuit board 70 is electrically connected to the air pump 610 and the solenoid valve 620. The breast pump is equipped with a power interface 50, which charges the battery 60 through an external power source. The battery 60 is electrically connected to the PCB circuit board 70, the air pump 610, and the solenoid valve 620.

The above contents are further detailed instructions to the disclosure in combination with the specific optimal embodiments, and it can not be concluded that the embodiments of the disclosure are limited to these instructions. For ordinary technicians in the technical field of the disclosure, without breaking away from the conception of the disclosure, the structure form can be flexible and changeable, and the series of products can be derived. Only a few simple deductions or replacements should be regarded in the protection scope of the present disclosure.

1. A leakage-proof wearable breast pump, comprising:
a milk bottle;
a front cover; and,
a negative pressure chamber;
said front cover and said negative pressure chamber are arranged above the milk bottle;
wherein a connecting chamber is provided between said front cover and said negative pressure chamber; said connecting chamber is provided above a bottle mouth of said milk bottle, a buffer chamber is provided between said milk bottle and said connecting chamber and communicated with said milk bottle and said connecting chamber respectively.

2. The leakage-proof wearable breast pump according to claim 1, wherein a top surface of said milk bottle extends upwards around said bottle mouth to form said buffer chamber; a bottom of said connecting chamber is provided with a cover plate to cover said buffer chamber; said cover plate is provided with a one-way valve, directly facing said bottle mouth; or
a bottom of said connecting chamber is provided with a cover plate, said cover plate extends downward to form said buffer chamber, said buffer chamber surrounds said bottle mouth; said cover plate is provided with a one-way valve, directly facing the bottle mouth; or
a bottom of said buffer chamber is connected with a top of said milk bottle and surrounds said bottle mouth, and a top of said buffer chamber is connected to a cover plate disposed on a bottom of said connecting chamber, said cover plate is provided with a one-way valve, directly facing said bottle mouth;
said plate cover is provided with a vent hole in communication with said buffer chamber and an outside.

3. The leakage-proof wearable breast pump according to claim 2, wherein an end of said one-way valve is close to or extends into said bottle mouth, and a gap is provided between said end of said one-way valve and an inner wall of said bottle mouth.

4. The leakage-proof wearable breast pump according to claim 1, wherein further comprising a mounting base with a bottom plate, said bottom plate is provided on a top of said milk bottle, said bottom plate is provided with an arch portion, and said connecting chamber and said buffer chamber are installed up and down in said arch portion.

5. The leakage-proof wearable breast pump according to claim 4, wherein said connecting chamber is provided with a guiding cylinder attached to said arch portion, a bottom of said arch portion is provided with an arc-shaped part, attached to a side wall of said buffer chamber.

6. The leakage-proof wearable breast pump according to claim 5, wherein said front cover is mounted on said mounting base and and is configures to match with a front side surface of said milk bottle to form a horn chamber; a small-opening end of said horn chamber is formed on said front cover and inserted into said arch portion, close to or adhered to one end of said guiding cylinder.

7. The leakage-proof wearable breast pump according to claim 5, wherein said negative pressure chamber includes a corrugated piece, a rear baffle, and a front baffle disposed at the other end of said guiding cylinder; said corrugated piece is made of a flexible silica gel material and comprises a corrugated portion and a side portion; a joint of said rear baffle and said front baffle clamps said side portion of said corrugated piece, thereby sealing a cavity between said rear baffle and said front baffle, and installing said corrugated portion in said negative pressure chamber.

8. The leakage-proof wearable breast pump according to claim 7, wherein said front baffle is provided with at least one hole, said negative pressure chamber is communicated with said connecting chamber and said horn chamber through said hole.

9. The leakage-proof wearable breast pump according to claim 4, wherein further comprising a negative pressure pump, said negative pressure pump includes an air pump, a solenoid valve, and a three-way valve; said three-way valve is disposed on said mounting base, and three air passages of said three-way valve are respectively in communication with said negative pressure chamber, said air pump, and said solenoid valve.

10. The leakage-proof wearable breast pump according to claim 4, wherein further comprising a rear cover mounted on said mounting base, said rear cover is matched with a rear side of said milk bottle to form an arc-shaped surface.
